(19) United States
(12) Patent Application Publication
(10) Pub. No.: US 2010/0022438 A1
(43) Pub. Date: Jan. 28, 2010

(54) Method of separating carbon nanotubes

(76) Inventors: Debdulal Roy, Twickenham (GB); Sanjib Bhattacharyya, Orleans (FR); Marie-Louise Saboungi, Orleans (FR); Jean-Paul Salvetat, Marcilly-en-Villete (FR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/311,265

(22) PCT Filed: Sep. 27, 2007
(86) PCT No.: PCT/GB2007/003683

§ 371 Date: Jul. 14, 2009

(30) Foreign Application Priority Data
Sep. 29, 2006 (GB) GB0619287.6

(51) Int. Cl. A61K 38/00; D01F 9/14; C07K 17/14; C07K 14/47

(52) U.S. Cl. 514/2; 423/447.1; 530/402; 530/356; 977/742; 977/847; 977/845; 977/915

(57) ABSTRACT

A method of separating carbon nanotubes having substantially the same diameter including the steps of: providing a sample of carbon nanotubes; separating individual nanotubes within the sample, and mixing with a solution comprising fibrous protein fibrils so that at least some individual nanotubes form a complex with the protein fibrils, and separating out those nanotubules which have formed a complex. Preferably the protein is collagen. The separated nanotubes can be used in the fields of electronics, medical and materials science.

The present invention relates to a method of separating carbon nanotubes according to their diameter, and to applications of separated carbon nanotubes.

A single wall carbon nanotube (SWCNT) is a rolled up structure of planar graphene sheet in the form of a cylinder. It is a one-dimensional nanostructure with semiconducting or metallic conductivity and technologically it is a very important material. Typically SWCNTs are grown by methods such as chemical vapor deposition, arc discharge, Laser ablation or hi pressure method. There are a number of commercial producers of single wall carbon nanotubes worldwide e.g. Carbon Nanotechnologies Incorporated (USA) [U.S. Pat. No. 6,761,870B1], Thomas Swan (UK), Nanocyl (Belgium) and Nanocarblab (Russia).

Such carbon nanotubes can be of various diameters ranging from about 0.5 nm to about 2 nm. Depending upon the way they are rolled up, they can have different chirality. The chirality of a single wall carbon nanotube determines its electronic and optical properties. Tubes of a specific diameter/chirality are required for many applications such as nanoelectronics, sensor technology and many fundamental materials research. However, it has not been possible to grow tubes with specific chirality or diameter [Kataura, H., Y. Kumazawa, Y. Maniwa, Y. Ohtsuka, R. Sen, S. Suzuki, and Y. Achiba, Diameter control of single-walled carbon nanotubes. Carbon, 2000. 38(11-12): p. 1691-1697]. There have been a few attempts to separate tubes according to their conducting properties (metallic and semiconducting) [M. Zheng, A. Jagota, M. S. Strano, A. P. Santos, P. Barone, S. G. Chou, B. A. Diner, M. S. Dresselhaus, R. S. Mclean, G. B. Onoa, G. G. Samsonidze, E. D. Semke, M. Usrey, and D. J. Walls, Structure-Based Carbon Nanotube Sorting by Sequence-Dependent DNA Assembly Science 2003 302: 1545-1548] [Maeda, Y., S. Kimura, M. Kanda, Y. Hirashima, T. Hasegawa, T. Wakahara, Y. F. Lian, T. Nakahodo, T. Tsuchiya, T. Akasaka, J. Lu, X. W. Zhang, Z. X. Gao, Y. P. Yu, S. Nagase, S. Kazaoui, N. Minami, T. Shimizu, H. Tokumoto, and R. Saito, Large-scale separation of metallic and semiconducting single-walled carbon nanotubes. Journal of the American Chemical Society, 2005. 127(29): p. 10287-10290, Chattopadhyay, D., I. Galeska, and P. Fotios, A Route for Bulk Separation of Semiconducting from Metallic Single-Wall Carbon Nanotubes. J. Am. Chem. Soc., 2003. 125: p. 3370-3375] and by diameter [K. H. An, Chol-Min Yang, J. Yeong Lee, C. Kang, J. H. Son, M. S. Jeong, Y. H. Lee, A diameter-selective chiral separation of single-wall carbon nanotubes using nitronium ions, J. of Electronic Materials, 2006, 35(2): p. 235-242] [M. S. Arnold, S. I. Stupp, M. C. Hersam, Enrichment of single-walled carbon nanotubes by diameter in density gradients, Nanoletters, 2005, 5(4): p 713-718]. However, some of the techniques separate by conductivity and those techniques which separate by diameter are limited to a specific conductivity of tubes (metallic/semiconducting) in a small range of selection diameter.

US 2005/0277675 describes the solubilisation of nanocarbons as a means of purification. Water soluble macromolecules are added to nanocarbons to form pseudomicelles which are then treated and dispersed by a homogenizer. The purified nanocarbons are removed by filtration. In this way impurities are eliminated. However, there is no separation of carbon nanotubes of a specific diameter.

The present invention seeks to provide an improved method of separating single wall carbon nanotubes of specific diameter.

According to a first aspect of the present invention, there is provided a method of separating carbon nanotubes having substantially the same diameter including the steps of:

providing a sample of carbon nanotubes of mixed diameter;

separating individual nanotubes within the sample;

mixing with a solution comprising protein fibrils so that at least some individual carbon nano tubules form a complex with said fibrils; and

separating out those nano tubules which have formed a complex.

To separate individual nanotubes within the sample, the tubes can be treated with acid and dispersed in water. Additionally or alternatively, surfactant can be added to the solution. In this case, the tubes do not need to be acid treated.

Typically the surfactant is SDS (sodium dodecyl sulphate), although other surfactants may be used.

In a preferred embodiment, the collagen is Type 1 collagen. The collagen may be obtained from calf skin. Other types of collagen may be used such as Types II, III, and/or VI. A mixture of different types of collagen can also be used.

Advantageously, the collagen is dissolved in water. However, other solvents may be used.

The step of separating out the tubules forming a complex may involve centrifugation and/or fractionation.

The sample may be acid treated prior to mixing with the surfactant and the collagen solution.

The diameter of the separated carbon nanotubules may be from about 0.8 to about 1.4 nm, preferably about 0.9 to about 1.3 nm, and more preferably about 1 to about 1.2 nm.

According to a second aspect of the present invention, there is provided a carbon nanotube substantially surrounded by protein fibrils.

According to a third aspect of the present invention, there is provided a carbon nanotube and fibrous protein complex, including a biosensor located substantially within the carbon nanotube.

According to a fourth aspect of the present invention, there is provided a combined preparation of fibrous protein and carbon nanotubes for use in therapy.

According to a fourth aspect of the present invention, there is provided use of a carbon nanotube and fibrous protein preparation for the manufacture of a medicament for the treatment of arthritis.

Preferred embodiments of the present invention will now be described by way of example only and with reference to the drawings in which:

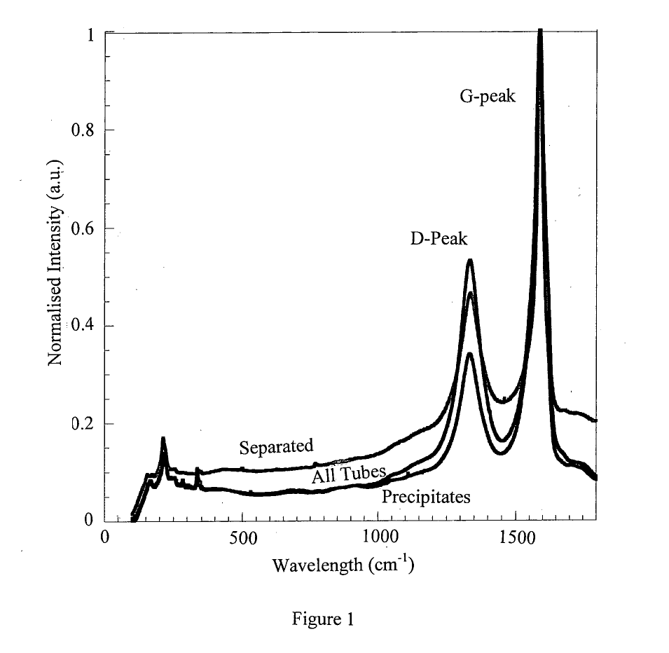

FIG. 1 shows raman spectra of the three samples of Nanocyl SWCNT. Raman measurements were performed with 633 nm excitation.

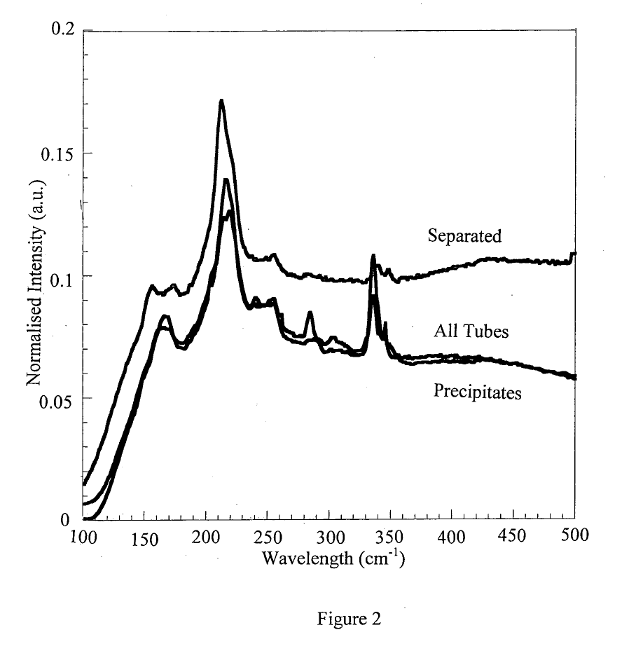

FIG. 2 shows radial breathing modes of Nanocyl SWCNT

$$( ( r = \frac{248}{ω} ) .$$

Raman measurements performed with 633 nm, using a Renishaw Raman spectrometer.

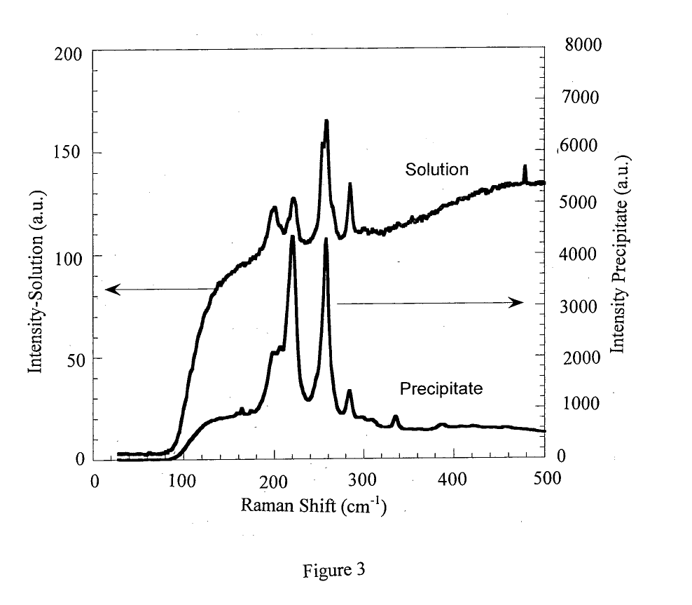

FIG. 3 shows radial breathing modes of Rice SWCNT from Rice. Raman measurements performed with 633 nm excitation using a Raman spectrometer.

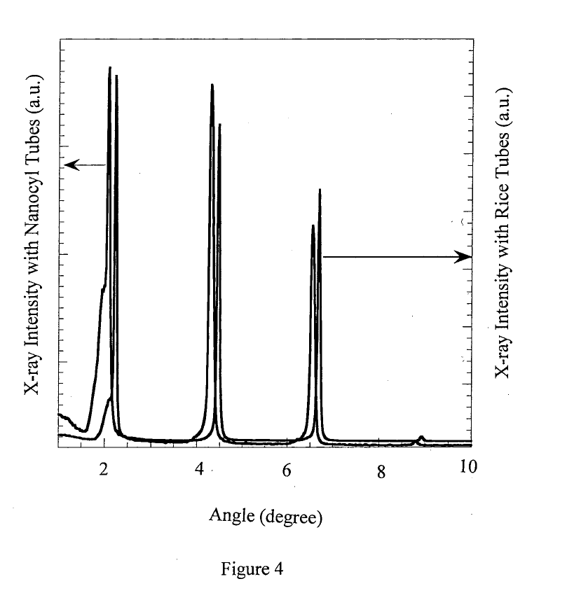

FIG. 4 shows x-ray diffraction results. With two types of tubes namely Nanocyl and Rice (supplied by carbon Nanotechnologies Incorporated, USA), it is observed from Raman spectroscopy that two diameters are selected. Selection of diameter of tubes (measured by Raman spectroscopy) produced different diameter of collagen micro-fibrils, measured by X-ray diffraction.

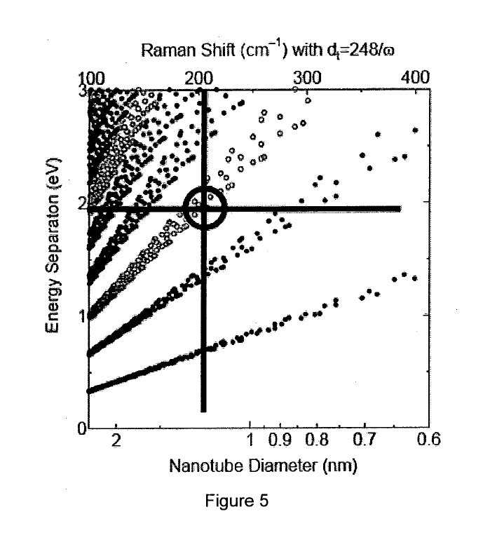

FIG. 5 shows Kataura plot showing the resonance region of the separated tubes. The black circles and grey circles are for semiconducting and metallic tubes respectively (Kataura, H., Y. Kumazawa, Y. Maniwa, I. Umezu, S. Suzuki, Y. Ohtsuka, and Y. Achiba, Optical Properties of Single Wall Carbon Nanotubes. Synthetic Metals, 1999. 103: p. 2555-2558).

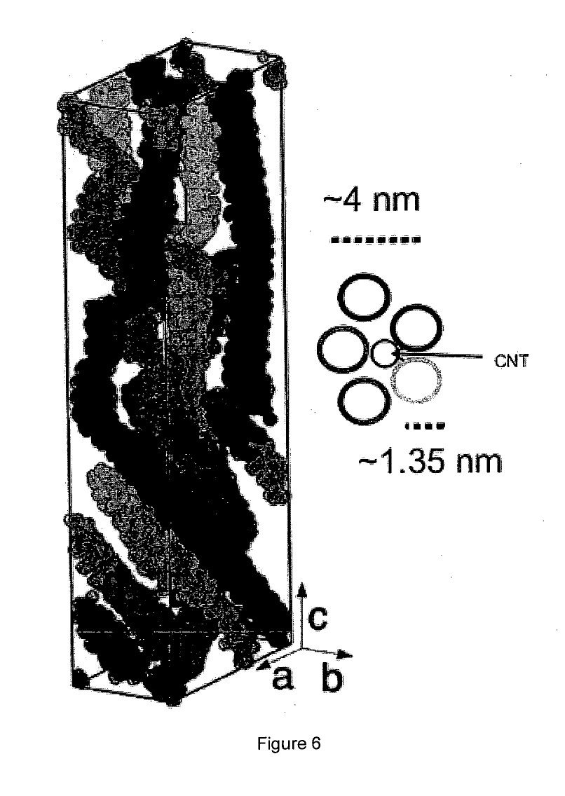

FIG. 6 shows a structure of a collagen micro fibril with a space for SWCNTs of about 1.15 nm diameter.

Raman spectroscopy is a powerful tool for characterizing carbon materials including diamond, graphite, diamond-like carbon, fullerenes and carbon nanotubes. In case of single wall carbon nanotubes, resonance Raman scattering takes place when the excitation laser energy matches with that of the band gaps. Therefore, the Raman intensity cannot be used to estimate the amount of specific tubes present in a sample [Rao, A. M., E. Richter, S. Bandow, B. Chase, P. C. Eklund, K. A. Williams, K. R. Subbaswamy, M. Menon, A. Thess, R. E. Smalley, G. Dresselhaus, and M. S. Dresselhaus, Diameter-Selective Raman Scattering from Vibrational Modes in Carbon Nanotubes. Science, 1997.275: p. 187-191]. In a Raman spectrum of SWCNT, there are two intense peaks between 1300 and 1600 cm⁻¹, and few peaks at low wave number regions (<400 cm⁻¹). The peak appearing at 1580 cm⁻¹ is called G-peak and that at ˜1350 cm⁻¹ is called D-peak. D peak is often associated to double resonance occurring due to presence of defects. The low wave numbers peaks are radial breathing modes which appear due to radial vibration of the tubes [Rao, A. M., E. Richter, S. Bandow, B. Chase, P. C. Eklund, K. A. Williams, K. R. Subbaswamy, M. Menon, A. Thess, R. E. Smalley, G. Dresselhaus, and M. S. Dresselhaus, Diameter-Selective Raman Scattering from Vibrational Modes in Carbon Nanotubes. Science, 1997. 275: p. 187-191]. The radial breathing mode frequency is dependent on the diameter of the tubes (d=248/wavenumber). The resonance behavior of single wall carbon nanotubes is complex and needs detailed analysis using the band structures of the tubes. Kataura et al [Kataura, H., Y. Kumazawa, Y. Maniwa, I. Umezu, S. Suzuki, Y. Ohtsuka, and Y. Achiba, Optical Properties of Single Wall Carbon Nanotubes. Synthetic Metals, 1999. 103: p. 2555-2558], proposed a plot which essentially explains the resonance Raman scattering characteristics of SWCNT (FIG. 4). In this work, a 1.9 eV excitation laser was used and the radial breathing mode is observed at around 210 cm⁻¹. The resonance region of our interest is marked by a circle in FIG. 4.

A typical SWNT/SDS/Collagen composite is synthesised in the following way: 24.0 mg of SWCNTs is sonicated in a bath sonicator (at 25 KHz) in 20 ml of 0.5% SDS solution in water for 1 hour. This disperses the SWCNTs, breaking the bundles and separating individual tubes. Subsequently 12 ml of collagen solution (2 mg/ml, collagen type I from calf skin, purchased from Sigma) is added to the above mixture and stirred for 24 hours at room temperature. The tubes interact with the collagen which forms microfibrils. SWCNTs of a suitable diameter become trapped within the collagen microfibrils (FIG. 6). In case not all of the tubes have been dispersed the mixture is separated by density. The mixture is sonicated for a further 30 minutes and centrifuged at 10,000 g for 25 minutes to get two distinct parts, one being supernatant and other being a precipitate. The supernatant comprises the separated tubes encased by collagen.

The tubes may be left encased with collagen or the collagen removed.

Since collagen is very unstable at higher temperatures, and burns off in an oxygen environment, it is straight forward to remove the collagen from the separated single wall carbon nanotubes for example by thermal treatment (such as in an oven at up to 500° C.) or chemical means (such as by acid treatment).

### EXAMPLE

Two sets of samples were prepared using two different sources of SWCNT:

A. Nanocyl tubes prepared by chemical vapour disposition (CVD); and
B. Rice tubes prepared by High Pressure Carbon Monoxide (HIPCO) process.
Some commercial preparations of SWCNTs contain metal nanoparticles and thus ideally should be cleaned, for example by acid treatment, prior to the separation method. The nanocyl tubes were purified via refluxing in 2 to 3 M HNO₃ solution for 12 to 48 hours (typically 24 hours), followed by vigorous centrifuging, repeated washing with deionized water, and drying under vacuum. This creates acid functionality mainly through carboxyl groups (—COOH) on the side-walls of the nanotubes. Optionally, the tubes may be subsequently treated with HCl.

SWNT/SDS/collagen composites were prepared using the method above for each sample.

The SDS coated tubes interact with collagen leading to the collagen becoming denatured. When the solution is stirred for 24 hours, some of the collagen fibrils tend to form a microfibril by self-assembling (FIG. 6). Individual collagen nanofibrils (diameter of ˜1.35 nm) arrange themselves in a quasi hexagonal arrangement with five fibrils. This has been shown by Orgel, J. P. R. O., T. C. Irving, A. Miller, and T. J. Wess, Microfibrillar Structure of Type I Collagen in situ. Biophysical Journal, 2006. 103(24): p. 9001-9005. Typically, the diameter of the microfibril assembly is about 3.8 nm and the diameter of the cavity in the microfibril assembly is 1.1 nm (as shown in FIG. 5). The SWCNT enters the cavity and is retained therein. Various values for the diameter have been reported in the literature, and therefore the calculated spacing will depend upon the value of the diameter of the microfibril and that of the nanofibril. We observe that with the two different sources of SWCNT, we see different diameter of the microfibrils, and the diameter of the micro-fibril is larger with the larger-diameter tube. This is because larger tubes need a bigger space within the microfibril and thus expand the diameter of the microfibril. If collagen is combined with surfactant in the absence of SWCNTs, the microfibril structure falls apart. However, a collagen nanotube composite structure can be formed with dispersed tubes without surfactant. For acid treated nanotubes, it is preferable to use surfactant.

Raman spectroscopy measurements were performed using a Renishaw Raman spectrometer with a 633 nm excitation laser. X-ray diffraction measurement was performed using standard equipment and technique.

### Sample Set A: Nanocyl Tubes

FIG. 1 shows the Raman spectra of three samples with Nanocyl tubes:

i) an acid treated sample (unseparated)

ii) a solution containing the separated tubes (following SDS and collagen treatment), and

iii) the precipitate containing all types of tubes.

The radial breathing modes (RBM) of the Raman spectra are shown in FIG. 2. It can be seen that the first sample (pure tubes) contain a number of RBM peaks. The precipitate also contains many RBM peaks. However, the solution has one RBM peak, meaning that the solution is rich in one diameter of tubes. The diameter is calculated to be about 1.2 nm.

### Sample Set B: Rice Tubes

FIG. 3 shows Raman spectra of samples prepared with Rice tubes:

i) separated tubes; and

ii) precipitated tubes.

It is very clear that the separated tube sample is richer with tube having RBM at around 250 cm⁻¹, which corresponds to diameter of about 1 nm. These tubes were not treated with acids before mixing with SDS.

X-ray diffraction results (FIG. 4) show the formation of regular structures similar to collagen micro-fibrils with both types of tubes. However, only the soluble part, and not the precipitate, shows a regular structure formation. The diameters of the collagen microfibril, measured from X-ray diffraction are 4.3 nm and 4 nm for Nanocyl and Rice tubes respectively. The diameters of the separated nanotubes are 1.2 nm and 1 nm for Nanocyl and Rice tubes respectively, and this difference is consistent with that measured from the radial breathing modes.

The present invention permits the separating of single wall carbon nanotubes of specific diameters using a simple, scalable and inexpensive technique, by exploiting the interaction of SWCNT and collagen. Raman spectroscopy provides evidence of diameter selection and x-ray diffraction provides evidence of micro-fibril formation.

Separated carbon nanotubes have a wide application in diverse fields including electronics, medicine and material science.

Instead of collagen it would be possible to use a similar interaction mechanism and choose different molecules to select the tubes of other diameters. It can also be used to deliver SWCNTs into the body which would be otherwise difficult due to their likely antigenicity. Since the SWCNT is wrapped in collagen, the SWCNT can be introduced into the body preventing it being attacked as a foreign body. Furthermore, material can be inserted inside the SWCNT before introduction. Biosensors for example can be loaded into SWCNTs and introduced into the body. Other materials can be loaded into SWCNTs, for example coloured material for use in cosmetics.

SWCNTs can be used to toughen skin, for example around scar tissue in order to prevent shrinkage of the skin. This is particularly useful in burns patients and following cosmetic surgery. Introducing SWCNTs into animal skin permits the skin to be toughened, improving the strength of the resulting leather.

Collagen is naturally present in many tissues around the body. One example is cartilage. This can be strengthened using SWCNTs which is advantageous where the cartilage has become worn or damaged (such as from arthritis) or in joint replacement surgery. A further example is bone marrow, whereby SWCNTs could be used as a seeding material.

Various modifications are contemplated.

Collagen of any kind and from any source can be used for separating tubes by the process described above. The collagen may be natural (from animal or human tissue) or synthetic.

The collagen may be modified in order to select a tube of different diameter.

Carbon nanotubes can be coated with a thin layer of organic or inorganic molecules in order to modify the effective diameter of the tube and thus enable selection of that diameter tube using a specific collagen.

Any separation process such as centrifugation or fractionation can be used to separate the solution part containing the separated tubes.

Any method of dispersing the nanotubes to obtain individual nanotubes can be used.

1. A method of separating carbon nanotubes having substantially the same diameter including the steps of:
providing a sample of carbon nanotubes;
separating individual nanotubes within the sample;
mixing the sample with a solution comprising protein fibrils so that at least some individual nanotubes form a complex with the protein fibrils, and
separating out those nanotubes which have formed a complex.

2. A method as claimed in claim 1, wherein the protein fibrils form an assembly having a cavity and wherein the complex comprises individual carbon nanotubes of a suitable diameter trapped within the cavity of the protein fibril assembly.

3. A method as claim in claim 1, wherein the protein is collagen.

4. A method as claimed in claim 3, wherein the collagen is type 1 collagen.

5. A method as claimed in claim 1, wherein the protein is dissolved in water.

6. A method as claimed in claim 1, wherein the step of separating individual nanotubes includes mixing the sample with a surfactant.

7. A method as claimed in claim 6, wherein the sample is acid treated prior to mixing with the surfactant.

8. A method as claimed in claim 6, wherein the surfactant is a solution of sodium dodecyl sulphate.

9. A method as claimed in claim 1 wherein the separation step includes centrifugation and/or fractionation.

10. A method as claimed in claim 1, wherein the mixing step involves sonicating the mixture.

11. A method as claimed in claim 10, wherein the mixture is sonicated at about 25 KHz.

12. A method as claimed in claim 1, further including the step of isolating the carbon nanotubes from the protein.

13. A method as claimed in claim 12, wherein the isolating step includes denaturing protein by heat or chemical means.

14. A method as claimed in claim 1, further including the step of measuring the diameter of the carbon nanotubes in the protein complex.

15. A method as claimed in claim 14, wherein the measuring step includes Raman spectroscopy.

16. Carbon nanotubes having substantially the same diameter obtainable obtained by a method as claimed in claim 1.

17. A carbon nanotube wrapped in a protein fibril assembly.

18. A carbon nanotube and fibrous protein complex, including a biosensor located substantially within the carbon nanotube.

19. (canceled)

20. (canceled)

21. (canceled)

22. (canceled)

23. A method of treating arthritis or of strengthening human or animal skin including administering to a subject in need thereof an effective amount of a combined preparation of fibrous protein and carbon nanotubes.
